(19) United States
(12) Patent Application Publication
(10) Pub. No.: US 2024/0164667 A1
(43) Pub. Date: May 23, 2024

(54) IRRITATION-LIMITING SENSOR FOR BODY MONITORING DEVICE

(71) Applicant: PKvitality, Paris (FR)

(72) Inventors: Luc PIERART, Villejuif (FR); Tracy PETIT, Montrouge (FR)

(73) Assignee: PKvitality, Paris (FR)

(21) Appl. No.: 18/282,681

(22) PCT Filed: Mar. 18, 2022
(86) PCT No.: PCT/FR2022/050501

§ 371 Date: Sep. 18, 2023

(30) Foreign Application Priority Data
Mar. 19, 2021 (FR) 2102766

(51) Int. Cl. A61B 5/145; A61B 5/00

(52) U.S. Cl. CPC A61B 5/14514; A61B 5/681; A61B 5/685; A61B 5/14532

(57) ABSTRACT

The invention relates to a sensor for a body monitoring device comprising:
an inner face (31) designed to come into contact with an individual's skin;
at least one micro-needle (32) arranged on said inner face (32);
at least one pad (34) arranged on the inner face of the substrate close to a microneedle and configured to absorb/adsorb moisture when the sensor (3) is on the skin.

### TECHNICAL FIELD

The invention concerns a body monitoring device and in particular a device for body monitoring by analysis of body fluid, typically interstitial fluid, by means of microneedle(s).

### STATE OF THE ART

Certain pathologies, such as diabetes, require daily monitoring of biochemical parameters in the human body, in particular concentrations of certain compounds (glycemia in the case of glucose).

For this purpose, it is common practice to prick a point on the skin so as to draw a drop of blood, and to analyze this drop either reactively (e.g. with a strip) or electronically (e.g. by at least one analytical sensor), so as to estimate the target parameter(s).

Today, we know of advanced, far less invasive systems that simply analyze the interstitial fluid, i.e. the fluid that fills the space between blood capillaries and cells. Its ionic composition is similar to that of blood plasma.

These advanced systems enable the desired biochemical parameters to be monitored transcutaneously, without the need for regular skin piercing and sampling.

In particular, a wrist-worn device called GlucoWatch was proposed, implementing a phenomenon called iontophoresis (or ionophoresis) in which an electric field is used to “draw” interstitial fluid through the skin to a sensor on the wall of the device. This concept was quickly abandoned, however, as only 6% of patients could tolerate the pain of electrical extraction. What's more, measurement results were unreliable.

Alternatively, transcutaneous probes have been proposed in the form of a self-adhesive patch that places a “sensor-micro-needle” just under the skin, so as to put the sensor in permanent fluid communication with the interstitial fluid, for continuous monitoring. Some of these patch-type transcutaneous probes include wireless communication means enabling interstitial fluid measurements to be transmitted to a mobile terminal, for storage and/or processing of measurements (verification of thresholds and variations, generation of statistics, triggering of alerts if necessary, etc.). Examples include the sugarBEAT™ and FreeStyle Libre systems.

Many of the systems on offer include a microneedle array. However, there is evidence that prolonged, immobile wearing of a microneedle array in contact with the skin leads to irritation and encourages the growth of bacteria.

### DESCRIPTION OF THE INVENTION

The invention overcomes at least one of these drawbacks.

To this end, the invention proposes a sensor for a body monitoring device comprising: an inner face intended to come into contact with the skin of an individual; at least one microneedle arranged on said inner face; at least one pad arranged on the inner face of the substrate close to a microneedle and configured to absorb/adsorb moisture when the sensor is on the skin.

The invention is advantageously completed by the following features, taken alone or in any technically possible combination thereof:
the pad is arranged between 0.1 and 10 mm from the microneedle;
it comprises a plurality of microneedles distributed on the inner face and a plurality of pads arranged on the inner face so that each microneedle is at a distance of between 0.1 mm and 10 mm from a pad;
it comprises a plurality of micro-needles distributed in a determined zone of the inner face, and comprising at least two pads, each arranged at the ends of said zone;
the pad is made of at least one absorbent material preferably selected from the following group: fiber, foam, textile fiber or artificial fiber, hydrogel, silica gel, magnesium sulfate, calcium chloride, calcium sulfate, lithium chloride, zeolites; or a non-woven pad preferably made of a mixture of viscose and polyester or a woven pad made of woven hydrophilic gauze;
the pad has a surface area of between 1 and 500 mm², preferably 15 mm² and/or a thickness of between 0.1 and 2 mm, preferably 0.5 mm;
the pad comprises one of the shapes selected from the following group: polygonal, rounded, parallelepiped, triangle, round;
it comprises at least one passage formed in the thickness of the sensor and in communication with the outside, said passage being configured to supply air to at least one microneedle, said passage preferably being permeable to water.
at least one passage is arranged between the microneedles;
it comprises at least one housing, at least one deck supporting at least one microneedle, each deck being fixedly mounted in a housing;
The invention also relates to a body monitoring device comprising a sensor according to the invention.

The addition of at least one absorbent pad helps to reduce irritation and thus limit the appearance of bacteria. In addition, it prolongs the wearing of the same sensor by increasing its duration of use. Users change their sensor less often.

### PRESENTATION OF FIGURES

Further features, purposes and advantages of the invention will be apparent from the following description, which is purely illustrative and non-limiting, and which should be read in conjunction with the appended drawings on which:

FIG. 1 illustrates a general view of a body monitoring device according to one embodiment of the invention;

FIGS. 2 and 3 show a bottom view of the sensor configurations of a monitoring device according to the invention.

FIG. 4 illustrates a microneedle surrounded by a channel used in a device according to the invention;

FIG. 5 illustrates a stage comprising four microneedles used in a device according to the invention.

On all the figures, similar elements bear identical references.

### DETAILED DESCRIPTION

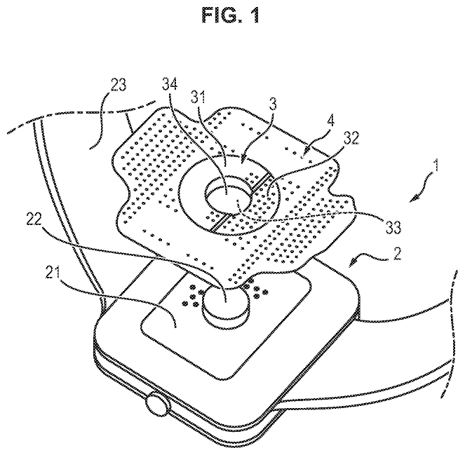

In relation to FIG. 1, a body monitoring device 1 comprises a case 2, a sensor 3 and an adhesive patch 4. Such a monitoring device is advantageously used to implement a body monitoring method.

Body monitoring means checking the biochemical constants of a person wearing the device, typically the concentration of a protein, hormone, marker, oxygen, nutrient, etc. in the person's body fluid. An example is blood glucose. If required, the skilled person can also monitor other physical body parameters such as temperature, hydration, etc.

The biochemical constant to be monitored is also considered to be the concentration of glucose (or glycemia) in the interstitial fluid of the skin. Interstitial fluid glycemia is considered to be representative of blood plasma glycemia.

The body fluid considered here is interstitial fluid, but other body fluids such as blood can also be considered.

Sensor 3 is a needle-type sensor designed to provide an electrical current measurement within the interstitial liquid of the carrier of device 1. The needles are advantageously arranged on an inner face 31 of the sensor 3. This inner face 31 is intended to be placed on the wearer's skin. The needles 32 are advantageously micro-needles. The sensor 3 preferably comprises between four and fifty microneedles, or even 400 microneedles. Of course, a different number can be considered without this limiting the description of the invention made here.

A microneedle is a needle with a small height, preferably between 10 μm and 1000 μm, more preferably between 0.3 mm and 0.8 mm. In addition, the microneedles are substantially pyramidal. The height of the microneedles is low enough to avoid contact with a wearer's nerve when the device is worn.

Micro-needles 31 are used to measure or sample body fluid.

Micro-needles 32 can be either hollow for fluid collection or solid for direct fluid analysis. In the case of liquid collection, the microneedles extract interstitial liquid from the dermis in a painless, bloodless way, and send it to a sensor housed in case 2. When liquid is to be analyzed, the microneedles take no liquid and incorporate the sensor on their surface in the form of a coating such as a biochemical material capable of reacting with the analysis to be carried out on the liquid.

Advantageously, the sensor 3 comprises a number of microneedles which consist of an array of microneedles in that they are electrically connected to one another. The microneedles pierce the skin to come into contact with the interstitial liquid when the sensor is in contact with the skin.

As shown in FIG. 1, the sensor 3 is joined to the adhesive patch 4, and together they can form a capsule. The sensor 3 may also be removable from the patch 4. Such a capsule is advantageously mounted removably with the case 2. In particular, the capsule, i.e. the sensor 3, preferably engages in a cavity 21 in the case 2, located on the side intended for contact with the skin. The sensor 3 also has an external face 34 opposite the internal face 31.

The sensor 3 shown here is circular in shape with a central hole 33, but can take other shapes: rectangular, oblong, ellipsoidal with or without a central hole. The central hole 33 allows the sensor 3 to be correctly positioned in the cavity 21 of the case 2 which includes a central stud 22.

The sensor 3 therefore comprises elements that allow the liquid to be sampled or the signals detected by each microneedle to be fed to the case 2 for processing (not described here).

The adhesive patch 4 is designed to stick to the skin and support the sensor 3, making it possible to detach the case 2 without removing the sensor 3, keeping it stuck to the body. Such a configuration makes it possible to avoid removing the sensor for certain operations involving only the case: battery recharging, repair, replacement, data extraction to a computer.

Case 2 is advantageously shaped like a watch case and comprises a bracelet 23 adapted to fit around a person's wrist. Case 2 houses several elements for analyzing or extracting interstitial fluid. In this respect, reference may be made to document WO 2019/141743 in the name of the Applicant, which describes in detail the measurement and detection of a physical quantity using micro-needles in contact with a body fluid that may or may not be sampled.

As you can see, the sensor is designed to be placed against the skin for several days, and in particular for more than three days.

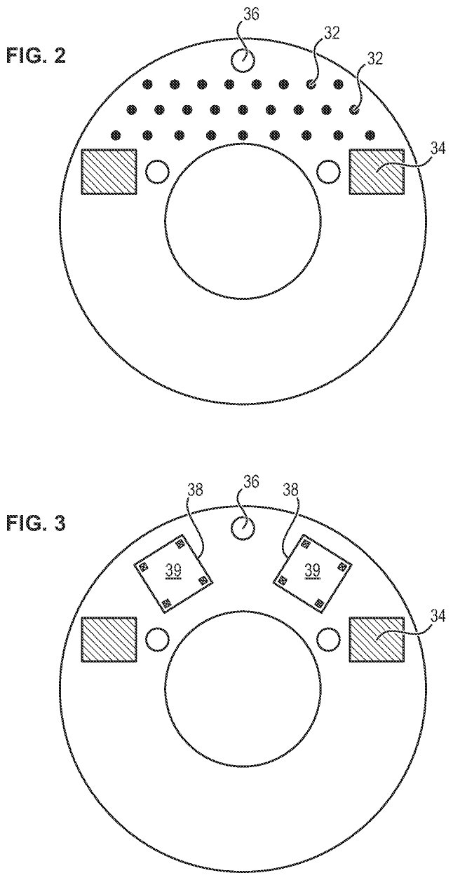

Advantageously, as shown in FIG. 2, the sensor 3 comprises at least one absorbent pad 34 arranged on the inner face 31 of the sensor 3. Such a pad 34 is configured to absorb moisture when the sensor 3 is on the skin. Preferably, the pad 34 is made of a moisture-absorbing, biocompatible material.

Pad 34 is made of a material selected from the following group: foam, textile fiber or artificial fiber, hydrogel, silica gel, magnesium sulfate, calcium chloride, calcium sulfate, lithium chloride, zeolites; or a non-woven pad preferably made of a mixture of viscose and polyester or a woven pad made of woven hydrophilic gauze.

This list is not exhaustive, and the skilled person will understand that any biocompatible material or combination of biocompatible materials can be used for the pad as long as it absorbs/adsorbs moisture.

Advantageously, the pad 34 is polygonal, in particular parallelepipedal, triangular, round, etc., the shape depending on how and where the pad 34 is arranged.

In addition, the pad has a surface area of between 1 mm 2 and 500 mm², preferably 15 mm² and/or a thickness of between 0.1 and 2 mm, preferably 0.5 mm.

Preferably, a pad 34 is positioned at a distance of between 0.1 and 10 mm from the microneedle.

In the case of several microneedles 32, several pads 34 are arranged on the inner face 31 of the sensor 3 and are distributed on this face 31 in such a way that each microneedle 32 is at a distance from the pad of between 1 and 10 mm.

This proximity ensures good absorption of moisture by the pad 34 during the period when the sensor 3 is positioned on the skin.

This not only limits irritation over a given period, but also increases the length of time the same sensor 3 is worn without the user feeling the need to change sensor 3.

In other words, the pad(s) 34 must be positioned as close as possible to each microneedle, without interfering with their operation. In fact, the pad(s) 34 must not touch the microneedles.

In a preferred embodiment, as illustrated in FIG. 2, the microneedles 32 are distributed over a specific zone 35 of the inner surface 31. In this case, absorbent pads 34 are arranged on either side of zone 35.

Such a distribution of micro-needles 32 is, for example, described in document WO 2020/025820 A1 to the Applicant.

When the sensor 3 has an oblong or circular or ellipsoidal shape, the microneedles are distributed over a sector of the inner face, with the pads 34 on either side of the sector (see FIG. 2, for example).

In a complementary manner, the sensor 3 comprises one or more passages 36 formed in its thickness, these passages 36 are in communication with the outside and are configured to bring air towards the face 31 in contact with the skin while being permeable to water. In this way, the air around the microneedles 32 and pads 34 is renewed, helping to dry out the area in contact with the sensor 3.

Preferably, passage 36 is formed across the entire thickness of sensor 3 from its outer face to the inner face.

Alternatively, the passage may extend from an area where the microneedles are located to an edge of the sensor 3, so that the passage is in contact with the outside. In this case, the passage is not formed over the entire thickness of the sensor, but only over part of it.

In any case, to be permeable to water, passage 36 may comprise a cross-section adapted to prevent the entry of water by capillary repulsion, or it may further comprise a barrier arranged in each passage 36, said barrier being made of a material permeable to water but not to air.

According to an embodiment illustrated in FIG. 3, a channel 37 surrounds the microneedles and may be in communication with a passageway. Such a channel is, for example, described in document WO 2021/023884 A1 to the Applicant.

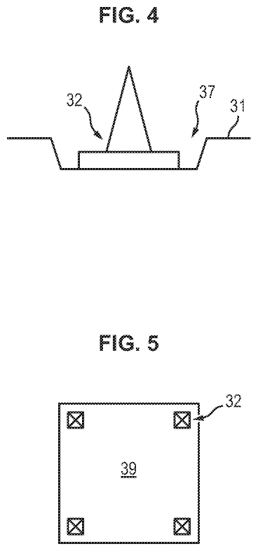

FIG. 4 illustrates a preferred embodiment in which the sensor 3 is ring-shaped (but may also be oblong) and comprises on its inner face housings 38 for accommodating decks 39, each of which supports at least one microneedle 32. As shown in FIG. 5, each deck 39 preferably supports four microneedles 32. The decks 39 are rectangular and the microneedles 32 are arranged at the four corners of one face of the deck 39.

As can be seen in FIG. 4, the sensor 3 comprises a central orifice 33 around which is delimited a sector in which the housings 38 and therefore the decks are distributed. At the ends of this sector are pads 34. In addition, passages 36 are distributed around this sector to enable air to be supplied to the microneedles 32.

At the upper end of the sector (furthest from the central orifice), the adhesive patch 4 surrounds the sensor 3 to enable it to be held in place on the individual's skin. In addition, a sealing ring 331 surrounds the central orifice 33.

1. A sensor (3) for a body monitoring device comprising:
an inner face (31) designed to come into contact with the skin of an individual;
at least one microneedle (32) arranged on said inner face (32);
at least one pad (34) of moisture-absorbing material, the pad (34) being arranged on the inner face of the sensor close to a microneedle so as to absorb moisture when the sensor (3) is on the skin.

2. The sensor of claim 1, in which the pad (34) is arranged between 0.1 and 10 mm from the microneedle.

3. The sensor of claim 1, comprising a plurality of microneedles distributed on the inner face (31) and a plurality of pads (34) arranged on the inner face so that each microneedle is at a distance of between 0.1 mm and 10 mm from a pad.

4. The sensor of claim 1, comprising a plurality of microneedles distributed in a determined zone of the inner face, and comprising at least two pads, each arranged at the ends of said zone.

5. The sensor of claim 1, wherein said pad consists of at least one absorbent material preferably selected from the following group: fiber, foam, textile fiber or artificial fiber, hydrogel, silica gel, magnesium sulfate, calcium chloride, calcium sulfate, lithium chloride, zeolites; or a non-woven pad preferably consisting of a mixture of viscose and polyester or a woven pad consisting of woven hydrophilic gauze.

6. The sensor of claim 1, wherein said pad has a surface area of between 1 and 500 mm², preferably 15 mm² and/or a thickness of between 0.1 and 2 mm, preferably 0.5 mm.

7. The sensor of claim 1, said pad comprises one of the shapes selected from the following group: polygonal, rounded, parallelepiped, triangle, round.

8. The sensor of claim 1, comprising at least one passage formed in the thickness of the sensor and in communication with the outside, said passage being configured to supply air to at least one microneedle, said passage preferably being permeable to water.

9. The sensor of claim 1, wherein said at least one passage is arranged between the microneedles.

10. The sensor of claim 1, comprising at least one housing, at least one deck supporting at least one microneedle, each deck being fixedly mounted in a housing.

11. A body monitoring device comprising a sensor according to claim 1.
